(19) United States
(12) Patent Application Publication
(10) Pub. No.: US 2008/0254053 A1
(43) Pub. Date: Oct. 16, 2008

(54) Protocol for treatment of diabetes

(76) Inventor: John P. Mullally, Ft. Gratiot, MI (US)

Correspondence Address: THE WEINTRAUB GROUP, P.L.C., SUITE 240, 32000 NORTHWESTERN HIGHWAY, FARMINGTON HILLS, MI, 48334, US

(21) Appl. No.: 12/157,807

(22) Filed: Jun. 13, 2008

Related U.S. Application Data
(63) Continuation-in-part of application No. 10/798,117, filed on Mar. 11, 2004.
(60) Provisional application No. 60/461,534, filed on Apr. 9, 2003.

(51) Int. Cl. A61K 39/00; A61K 31/47; A61K 31/58; A61K 31/445

(52) U.S. Cl. 424/193.1; 514/311; 424/184.1; 514/174; 514/317

(57) ABSTRACT

A method and composition for reducing highly sensitive C-reactive protein is provided for the treatment of Type I and Type II diabetes which is achieved through the daily administration of a leukotriene inhibitor, an antihistamine and a corticosteroid. The composition may be administered singly or as a single medicament. Typically, the leukotriene inhibitor and antihistamine are administered orally and the steroid nasally infused.

### CROSS-REFERENCE TO RELATED APPLICATIONS

This application is a continuation-in-part application of co-pending U.S. patent application Ser. No. 10/798,117 filed on Mar. 11, 2004 entitled “Composition and Method for Treating Inflammations” which, in turn, is a completion application of Provisional Patent Application Ser. No. 60/461,534 filed on Apr. 9, 2003, the entire disclosures of which are hereby incorporated by reference.

### BACKGROUND OF THE INVENTION

1. Field of the Invention

The present invention pertains to medical protocols. More particularly, the present invention concerns medical protocols for the treatment of diabetes.

2. Description of the Prior Art

The diseases that plague mankind are the body's inflammatory response to the air we breathe and the food and liquids we ingest. This inflammatory response occurs in the nasal, sinus, and mouth cavities, as well as the gastrointestinal tract. It transpires in the form of phlegm or mucous congestion which enters the bloodstream and is then delivered to the inflammatory pathways of the body's organs and blood vessels. Mucous is transmitted systemically in a similar manner as that of inhaled insulin. Currently the only measure of systemic inflammation is known as a High Sensitivity C-reactive Protein test, or HSCRP.

Since the body's systemic inflammation acts in response to the air, food, and liquids that enter our bodies, the state of the environment is of rapidly growing concern. The levels of greenhouse gases, such as carbon dioxide, continue to rise. Pollen counts rise proportionately with carbon dioxide levels. Therefore pollen counts are rising as well. The increase in environmentally-induced pollen counts has contributed to the current epidemic of allergic and non-allergic rhinitis throughout the world. Both the allergic and non-allergic formation of mucous is transmitted systemically causing diseases such as diabetes. Inflammation also shuts down one's metabolism, leading to obesity. Additionally, inflammation causes genetic changes in our DNA which triggers the formation of disease, and makes future generations more prone to disease at young ages.

In the above-referred to co-pending application, the disclosure of which is hereby incorporated by reference, there is disclosed therein a medical protocol for lowering C-reactive protein in the body of a user. According to the protocol defined therewithin, it is theorized that by eliminating sinus drainage the bacteria and viruses which are normally captured therewithin and which ultimately enter into the bloodstream are eliminated through the administration of a leukotriene inhibitor, an antihistamine and a steroid. Levels of highly sensitive C-reactive protein are reduced in the body of the user, thus, relieving systemic inflammation. The principal component of the protocol is the leukotriene inhibitor. However, by the administration of all three of the components of the protocol, dramatic decreases in the C-reactive protein in the system of a user has been effected thereby providing relief from a variety of systemic inflammations.

To this end, it has now been observed that by lowering C-reactive protein levels in the body of a user, there is a concomitant improvement in the condition of those suffering from diabetes. Thus, the present invention adopts and adapts the protocol of the above-referred to co-pending application in the treatment of diabetes.

The medical protocol described herein prevents the inflammatory response to the air, food, and liquids with which the body comes into contact. By reducing the inflammatory response, diabetes in a person can be prevented or regressed. This is a new philosophy in approaching medicine.

### SUMMARY OF THE INVENTION

In accordance with the present invention, there is provided a protocol for the treatment of diabetes by the lowering of C-reactive protein within the body of such user. The protocol hereof, generally, comprises the administering to a user a daily dosage of: (a) a leukotriene inhibitor, (b) an antihistamine, (c) a corticosteroid, and (d) mixtures thereof.

In a second aspect hereof there is provided a composition for reducing C-reactive protein for the treatment of diabetes which comprises an admixture of a leukotriene inhibitor, an antihistamine and a corticosteroid.

The composition is administered on a daily basis as a preventative treatment.

For a more complete understanding of the present invention reference is made to the following detailed description and accompanying non-limitative example.

### DESCRIPTION OF THE PREFERRED EMBODIMENTS

As hereinabove noted, the present invention comprises, in a first aspect, a method for reducing and/or eliminating C-reactive protein in the body to treat diabetes of a user by administering a composition selected from the group consisting of: (a) a leukotriene inhibitor, (b) an antihistamine, (c) a corticosteroid, and (d) mixtures thereof.

In a second aspect hereof there is provided a composition for treating diabetes by reducing C-reactive protein which comprises an admixture of at least two of and, preferably, each of: (a) a leukotriene inhibitor, (b) an antihistamine, and (c) a corticosteroid.

In accordance with the present invention it has been found that a daily treatment of the above-noted composition is effective in reducing highly sensitive C-reactive protein and the concomitant diabetic conditions.

Generally, the leukotriene inhibitor is administered in a dosage from about 1 to 20 milligrams on a daily basis and, preferably, from about 5 to about 15 milligrams. The leukotriene inhibitor may be ingested as a pill, capsule, as a liquid, etc.

Similarly, the antihistamine is ingested, orally or nasally, on a daily basis and in an amount ranging from about 50 to about 250 milligrams and, preferably, from about 175 to about 200 milligrams daily.

The corticosteroid is usually found in a liquid transport or delivery medium, such as a nasal spray or the like and ordinarily, a minimal amount ranging from about 110 μcg to about 220 μcg as obtained from about 1 to about 4 nasal sprays is effective once or more daily.

In using this treatment, it is preferred where all three medicaments are used and that the leukotriene inhibitor and the antihistamine be administered each as a pill or gel cap while the steroid, as noted, is infused as a nasal spray.

Typical of the leukotriene inhibitors are those which are selected from the group consisting of albuterol sulfate, aminophylline, amoxicillin, ampicillin, astemizole, attenuated tubercle bacillus, azithromycin, bacampicillin, beclomethasone dipropionate, budesonide, bupropion hydrochloride, cefaclor, cefadroxil, cefixime, cefprozil, cefuroxime axetil, cephalexin, ciprofloxacin hydrochloride, clarithromycin, clindamycin, cloxacillin, doxycycline, erythromycin, ethambutol, fenoterol hydrobromide, fluconazole, flunisolide, fluticasone propionate, formoterol fumarate, gatifloxacin, influenza virus vaccine, ipratropium bromide, isoniazid, isoproterenol hydrochloride, itraconazole, ketoconazole, ketotifen, levofloxacin, minocycline, montelukast sodium, moxifloxacin, nedocromil sodium, nicotine, nystatin, ofloxacin, orciprenaline, oseltamivir, oseltamivir sulfate, oxtriphylline, penicillin, pirbuterol acetate, pivampicillin, pneumococcal conjugate vaccine, pneumococcal polysaccharide vaccine, prednisone, pyrazinamide, rifampin, salbutamol, salmeterol xinafoate, sodium cromoglycate (cromolyn sodium), terbutaline sulfate, terfenadine, theophylline, triamcinolone acetonide, zafirlukast, zanamivir, and the like, as well as mixtures thereof.

The inflammation reducing leukotriene inhibitor may be any of those commercially available leukotriene inhibitors such as “Zyflo®” (zileuton), “Accolate®” (zafirlukast), and “Singulair®” (a montelukast sodium) each sold commercially and available in pill form. Preferably, the leukotriene inhibitor is the montelukast sodium, which is sold commercially in pill form under the trademark “SINGULAIR®.”

The antihistamine can be any of those which are commercially available such as those sold under the name “Zyrtec®” (cetirizine), “Allegra®” (fexofenadine), “Claritin®” (loratadine), and “Clarinex®” (desloratadine).

The nasal steroid is, preferably, triamcinolone acetoniode, sold commercially under the name “Nasacort AQ®.” Other useful nasal steroids are those sold commercially under various trademarks such as, for example, “Flonase®” (fluticasone propionate), “Asteling” (azelastine), “Nasonex®” (mometasone furoate monohydrate), and “Rhinocort Aqua®” (budesonide), to name a few.

According to the invention, a preferred dosage or “composition” to treat diabetes includes at least two of and, preferably, all three of: (a) a capsule of Singulair®, (b) a capsule of Allegra®, and (c) a prescribed infusion of Nasacort AQ® into each nasal passage. By taking the daily dosages, it is possible to reduce highly sensitive C-reactive protein and, thus, improve the user's diabetic condition.

Although not wishing to be bound any theory, it is believed that the inflammation of the sinuses and nasal pathways is transmitted systemically through a bacteria, virus, fungus or an immune response associated with sinus drainage that is, then, transmitted through the bloodstream. It is believed that this theory also applies to other pathways including arteries, veins, and to some degree, body organs. What the present invention contemplates is the blocking of the inflamed pathways and, thus, treating the condition.

It is contemplated that the medicaments defined herein be administered at one time. It is also contemplated and within the scope hereof, that a single compound, as a liquid vehicle, be administered with the requisite amounts. In other words, there would be provided as a composition a sprayable compound having the leukotriene inhibitor, the antihistamine, and the corticosteroid all suspended in a liquid vehicle or non-toxic delivery system which can then be administered either orally or nasally through a spray and transmitted either through the mouth or the nasal passages.

Alternatively, it is possible to provide a capsule, pill or gel cap containing at least two, or preferably, all three of the components hereof.

In attempting to appreciate the benefits of the present invention, it is worthy to note that bacteria, fungi, and viruses all colonize in the sinus cavity. By attacking the source those diseases which are attributable to elevated high sensitive C-reactive protein levels in the blood are, necessarily, dramatically reduced and/or eliminated.

Following the present protocol, the hemoglobin HgA^{IC} values were 7.2 and 7.5, respectively, for two patients. As such, blood sugar was more normal, obviating the need in some cases for reliance on diabetes medicaments. Importantly herein, the protocol reduced the HgA^{IC} value for one of the patients by 20.2%, from a high of 9.4. Preferably, the American Diabetes Association recommends that the hemoglobin AIC value should be less than 7%, and that treatment be pursued for patients when the value is consistently above 8%.

It is further theorized that the present method may also overcome the systemic inflammation that precludes pancreatic cells from producing insulin, thereby, potentially overcoming what is identified as Type I diabetes, as noted hereinabove. The present method is believed useful in lowering blood pressure in patients with diabetes and persons with renal insufficiency.

As is known to those skilled in the art, typically a Type I diabetes sufferer is administered an antibiotic-antifungal pharmaceutical combination to treat a chronic infection. The present method can be used in conjunction with these antibiotics and antifungals for treating chronic infections.

In practicing the present invention, depending on the type of condition of the patient to be treated and the severity, ordinarily improvements can be seen in anywhere from two days to about one month on.

Because of the nature of the medicaments, i.e. all over-the-counter compositions, they can be administered to most persons safely and, typically, without interference with other medicines which may be taken until the full effects of the present invention are realized. Following is a non-limitative illustrative example further showing the efficacy of the present invention.

### EXAMPLE

The present protocol was administered to three patients. Specifically, each patient was administered once daily: (a) one hundred eighty (180) milligrams of an antihistamine, sold under the mark Allegra®; (b) ten (10) milligrams of a leukotriene inhibitor, sold under the mark Singulair®; and (c) two (2) nasal sprays in each nostril of a nasal steroid, sold under the mark Nasacort AQ®. The results from these tests are shown below in Table 1.

In Table 1 below, the following patient parameters were observed and recorded:

HgA^{IC} hemoglobin, %, and final value (in parenthesis); Chol.: Cholesterol,mg/dL; Tri.: Triglycerides, mg/dL; LDL, mg/dL; HDL, mg/dL; Ratio of Cholesterol/HDL and final value of ratio (in parentheses); CRP: c-reactive protein, mg/dL.

The Parameters report a % change in the parameter, the + and − indicate whether the change was an increase/decrease, and NA indicates that data is “not available.”

TABLE 1

| | | | | | | | Chol. | |
|---|---|---|---|---|---|---|---|---|
| | PATIENT RESULTS | HgA^{IC} | Chol. | Tri. | LDL | HDL | HDL | CRP |
| 1 | Timothy, Age 52, on | NA | NA | NA | NA | NA | NA | −34.6 |
| | NAS protocol 6 weeks. | | | | | | | (4.9) |
| | Results: beneficial for | | | | | | | |
| | diabetes condition, | | | | | | | |
| | HgA^{IC} lowered and CRP | | | | | | | |
| | down, and taken off | | | | | | | |
| | Avandia during NAS | | | | | | | |
| | protocol. | | | | | | | |
| 2 | William. Age 70, NAS | −20.5 | +44.34 | −43.0 | +59.7 | +56.5 | −8.0 | NA |
| | protocol beneficial for | (5.4) | | | | | (2.3) | |
| | diabetes condition. | | | | | | | |
| 3 | Edward, Age 17, a Type | 8.9 | 169 | 98 | 101 | 48 | NA | 0.29 |
| | I diabetic. After 5 weeks | | | | | | | |
| | of treatment showed | | | | | | | |
| | evidence of producing | | | | | | | |
| | his own insulin; went | | | | | | | |
| | from 0.002 to 14 Iu/ml | | | | | | | |
| | without wearing an | | | | | | | |
| | insulin pump. | | | | | | | |

From the above it is to be readily seen that the diabetic patients were afforded an effective treatment.

It should be noted that the present invention also provides a new perception pertaining to how the body ages and acquires disease for both humans and animals alike. Although not wishing to be bound by any theory, it is known that sugar intake, in the form of candy, pop, and desserts, cause mucous formation in the sinuses, mouth, and nasal cavities, as well as the gastrointestinal tract, thereby causing diseases and aging, including, elevated glucose levels in diabetics. Breathing pollution, pollen, dirt, dust, humidity changes, smoke, etc. cause mucous formation in the sinuses, mouth, and nasal cavities, as well as the gastrointestinal tract. In turn the mucous formation causes disease and aging.

The medical protocol described herein prevents the inflammatory response to the air, food, and liquids with which the body comes into contact. By reducing the inflammatory response, diabetes in a person can be prevented or regressed. This is a new philosophy in approaching medicine. Although the invention described herein is directed towards a diabetic indication, it is believed it will also potentially be used for preventing and treating cancer, Alzheimer's, heart disease, cardiovascular disease, peripheral vascular disease, arthritis (all types), seizures, Parkinsonism, inflammatory bowel disease, prevention of common colds, asthma, chronic obstructive pulmonary disease, and all lung disease. It is also theorized that it may be used for anti-aging by treating loss of vision, loss of hair color, wrinkling of skin, varicose vein formation, periodontal disease, and so forth.

Having, thus, described the invention, what is claimed is:

1. A method for treating systemic inflammation by reducing highly sensitive C-reactive protein levels in the body of a user which comprises:
administering on a daily basis for a period of at least about 2 days, a composition selected from the group consisting of
(a) a leukotriene inhibitor,
(b) an antihistamine,
(c) a corticosteroid, and
(d) mixtures thereof.

2. A method for the treatment of diabetes comprising:
administering on a daily basis for a period of at least about 2 days, a composition selected from the group consisting of
(a) a leukotriene inhibitor,
(b) an antihistamine,
(c) a corticosteroid, and
(d) mixtures thereof
wherein highly sensitive C-reactive protein levels in the body of a user are reduced.

3. The method of claim 2 wherein the selected composition is used in an amount of
(a) from about 1 to about 20 milligrams of leukotriene inhibitor,
(b) from about 50 to about 250 milligrams of antihistamine, and
(c) from about 110 μcg to about 220 μcg of corticosteroid.

4. The method of claim 3 wherein the selected composition is used in an amount of:
(a) from about 5 to about 15 milligrams of the leukotriene inhibitor,
(b) from about 175 to about 200 milligrams of the antihistamine, and
(c) from about 110 μcg to about 220 μcg of the corticosteroid.

5. The method of claim 3 wherein the leukotriene inhibitor is selected from the group consisting of:
albuterol sulfate, aminophylline, amoxicillin, ampicillin, astemizole, attenuated tubercle bacillus, azithromycin, bacampicillin, beclomethasone dipropionate, budesonide, bupropion hydrochloride, cefaclor, cefadroxil, cefixime, cefprozil, cefuroxime axetil, cephalexin, ciprofloxacin hydrochloride, clarithromycin, clindamycin, cloxacillin, doxycycline, erythromycin, ethambutol, fenoterol hydrobromide, fluconazole, flunisolide, fluticasone propionate, formoterol fumarate, gatifloxacin, influenza virus vaccine, ipratropium bromide, isoniazid, isoproterenol hydrochloride, itraconazole, ketoconazole, ketotifen, levofloxacin, minocycline, montelukast sodium, moxifloxacin, nedocromil sodium, nicotine, nystatin, ofloxacin, orciprenaline, oseltamivir, oseltamivir sulfate, oxtriphylline, penicillin, pirbuterol acetate, pivampicillin, pneumococcal conjugate vaccine, pneumococcal polysaccharide vaccine, prednisone, pyrazinamide, rifampin, salbutamol, salmeterol xinafoate, sodium cromoglycate (cromolyn sodium), terbutaline sulfate, terfenadine, theophylline, triamcinolone acetonide, zafirlukast, zanamivir, and mixtures thereof.

6. The method of claim 3 wherein the antihistamine is selected from the group consisting of:
cetirizine, fexofenadine and loratadine.

7. The method of claim 3 wherein the steroid is selected from the group consisting of:
mometasone furoate mononhydrate, triamcinalone, acetoniode, budesonide, fluticasone propionate and azelastine.

8. The method of claim 3 wherein:
(a) the leukotriene inhibitor is montelukast sodium,
(b) the antihistamine is selected from the group consisting of cetirizine, fexofenadine and loratadine, and
(c) the steroid is selected from the group consisting of azelastine and fluticasone propionate.

9. The method of claim 3 wherein the composition comprises:
(a) the leukotriene inhibitor,
(b) the antihistamine, and
(c) the corticosteroid.

10. The method of claim 3 wherein:
the leukotriene inhibitor and the antihistamine are administered orally and the steroid is nasally infused.

11. A composition for reducing C-reactive protein for the treatment of diabetes, consisting essentially of:
(a) a leukotriene inhibitor,
(b) an antihistamine, and
(c) a corticosteroid.

12. The composition of claim 11 wherein the composition comprises:
(d) from about 1 to about 20 milligrams of the leukotriene inhibitor,
(e) from about 150 to about 250 milligrams of antihistamine, and
(f) from about 110 μcg to about 220 μcg of corticosteroid.

13. The composition of claim 12 wherein the composition comprises:
(d) from about 5 to about 15 milligrams of leukotriene inhibitor,
(e) from about 175 to about 200 milligrams of antihistamine, and
(f) from about 10 μcg to about 220 μcg of corticosteroid.

14. The composition of claim 12 wherein the leukotriene inhibitor is selected from the group consisting of:
albuterol sulfate, aminophylline, amoxicillin, ampicillin, astemizole, attenuated tubercle bacillus, azithromycin, bacampicillin, beclomethasone dipropionate, budesonide, bupropion hydrochloride, cefaclor, cefadroxil, cefixime, cefprozil, cefuroxime axetil, cephalexin, ciprofloxacin hydrochloride, clarithromycin, clindamycin, cloxacillin, doxycycline, erythromycin, ethambutol, fenoterol hydrobromide, fluconazole, flunisolide, fluticasone propionate, formoterol fumarate, gatifloxacin, influenza virus vaccine, ipratropium bromide, isoniazid, isoproterenol hydrochloride, itraconazole, ketoconazole, ketotifen, levofloxacin, minocycline, montelukast sodium, moxifloxacin, nedocromil sodium, nicotine, nystatin, ofloxacin, orciprenaline, oseltamivir, oseltamivir sulfate, oxtriphylline, penicillin, pirbuterol acetate, pivampicillin, pneumococcal conjugate vaccine, pneumococcal polysaccharide vaccine, prednisone, pyrazinamide, rifampin, salbutamol, salmeterol xinafoate, sodium cromoglycate (cromolyn sodium), terbutaline sulfate, terfenadine, theophylline, triamcinolone acetonide, zafirlukast, zanamivir, and mixtures thereof.

15. The method of claim 3 wherein the antihistamine is selected from the group consisting of:
cetirizine, fexofenadine and loratadine.

16. The composition of claim 12 wherein the steroid is selected from the group consisting of:
(a) mometasone furoate mononhydrate,
(b) triamcinalone,
(c) acetoniode,
(d) budesonide
(e) fluticasone propionate, and
(f) azelastine.

17. The composition of claim 12 wherein:
(a) the leukotriene is montelukast sodium,
(b) the antihistamine is selected from the group consisting of cetirizine, fexofenadine and loratadine, and
(c) the steroid is selected from the group consisting of azelastine and fluticasone propionate.

18. The composition of claim 17 wherein:
the leukotriene and the antihistamine are administered orally and the steroid is nasally infused.

19. The method of claim 3 wherein:
(a) the leukotriene inhibitor is a montelukast sodium, the inhibitor being used in an amount ranging from about 5 to about 15 milligrams,
(b) the antihistamine is selected from the group consisting of cetirizine, fexofenadine and lortadine, the antihistamine being used in an amount ranging from about 175 to about 200 milligrams, and
(c) the steroid is selected from the group consisting of:
mometasone furoate monohydrate, triamcinalone, acetoniode, budesonide, fluticasone propionate, and azelastine, the steroid being used in an amount ranging from about 110 μcg to about 220 μcg.
